(19) United States
(12) Patent Application Publication
(10) Pub. No.: US 2024/0374533 A1
(43) Pub. Date: Nov. 14, 2024

(54) COMPOSITIONS AND METHODS FOR TREATING PEDICULOSIS

(71) Applicant: MIRADOR MEDICAL TECHNOLOGIES LLC, Austin, TX (US)

(72) Inventor: Kelly TJELMELAND, Austin, TX (US)

(73) Assignee: MIRADOR MEDICAL TECHNOLOGIES LLC, Austin, TX (US)

(21) Appl. No.: 18/684,512

(22) PCT Filed: Aug. 16, 2022
(86) PCT No.: PCT/US2022/075020

§ 371 Date: Feb. 16, 2024

Related U.S. Application Data
(60) Provisional application No. 63/234,650, filed on Aug. 18, 2021.

(51) Int. Cl. A61K 31/045; A61K 9/107; A61K 47/18; A61K 47/20; A61P 33/14

(52) U.S. Cl. CPC A61K 31/045; A61K 9/107; A61K 47/186; A61K 47/20; A61P 33/14

(57) ABSTRACT

The present invention relates generally to methods of use and compositions for treatment of lice infestations, particularly head lice infestations and the eggs, larvae, and/or adults lice that cause pediculosis. The composition includes an oil-in-water emulsion capable of treating pediculosis by suffocation of lice, including eggs, larvae, and/or adult lice.

### CROSS REFERENCE TO RELATED PATENT APPLICATIONS

This application claims the benefit of priority to U.S. Provisional Patent Application Ser. No. 63/234,650, filed Aug. 18, 2021, hereby incorporated by reference in its entirety.

### BACKGROUND OF THE INVENTION

### A. Field of the Invention

The present invention relates generally to the treatment of parasite infestations of the human body, and more particularly, but not by way of limitation, to methods and compositions to treat the eggs, larvae, and/or adults of lice that cause pediculosis.

### B. Description of Related Art

Pediculosis capitis (head lice) is an infestation of a subspecies of Pediculosis humans, a species of louse that infects the head of humans, and persist in developed countries and underdeveloped countries, despite the availability of modern chemical insecticide treatments, public health education, and community based programs of lice eradication. The insect is transmitted to non-infected individuals through shared clothing and hairbrushes. Once present on the head, the adult female louse has a lifespan of about 25 days and lays up to 10 eggs each day, which are attached to the hair shaft. Eggs hatch within 7-9 days, producing more adult lice which continue the cycle. As the lice feed, they inject their digestive juices and fecal material into the skin causing pruritus or itching of the skin.

The persistence of lice in developed countries, as well as underdeveloped countries, is due to a combination of factors. For example, some lice control programs suffer logistical problems (e.g., limited access to clinics or school-based programs where affected individuals may be treated). Additionally, some chemical treatments are not entirely effective, particularly shampoo formulations which can be largely diluted during use.

Other commonly used pediculicidal compositions rely on residual action on the hair by the pesticide after application to the affected area of the patient. For example, permethrin cream rinses (e.g., NIX®) are applied after shampooing and rely on the residual action of the insecticide on the hair after the shampoo treatment, as noted by Taplin et al. (Taplin, D., Meinking, T. L., Castillero, P. M., Sanchez, R., Permethrin 1% creme rinse for the treatment of Pediculus humanus var. capitis infestation, Pediatr. Dermatol. 3(4): 344-348 (1986)). However, despite the “over-the-counter” convenience of residually active insecticides, such as permethrin, for some consumers, the effect may be inconsistent in development or reduced by environmental conditions resulting in treatment failures as evidenced by the emergence of louse eggs after treatment. In addition, because these cream rinses are applied after shampooing, many of the lice are not physically dislodged from their reflex action of grasping the hairs. Accordingly, dead lice may only be found during subsequent washing and/or extensive grooming. The main disadvantage of residual action-based pediculicides is that as residual activity wears off, there will be a level at which point any lice invading a person's head will not be killed by the lower pediculicidal levels. Some have postulated that these conditions have led to resistant strains of lice commonly called “super lice.”

Pyrethrins, a class of organic compounds derived from Chrysanthemum cinerariifolium, are also commonly used as active ingredients in pediculicides and demonstrate a good safety level (e.g., RID® lice killing shampoo, Triple X). However, most of these compositions are shampoos which, due to their presentation as discussed above, show minimal activity against louse eggs (Burgess, I. Malathion lotions for head lice a less reliable treatment than commonly believed. Pharm. J. 247: 630-632 (1991)). Natural pyrethrins have long been successful as insecticides because they rapidly incapacitate insects, an effect known as “knockdown”. Provided that sufficient pyrethrins are present, the knockdown effect persists to the eventual death of the insect. The lethal effect of pyrethrins is normally synergized by adding piperonyl butoxide to incapacitate the enzymes that would otherwise detoxify the pyrethrins.

However, the rapid penetration and action of pyrethrins on lice can be hindered if lice are exposed to water. Lice which come in contact with water grasp hairs reflexively and close their breathing spiracles to avoid being drowned. Since insecticides are effective only because of their entry into the lice via the spiracles, when these spiracles are closed, the chances of the insecticide penetrating the insect when the lice is first, or simultaneously, exposed to water is negligible, especially if the formulation is a shampoo rather than a residual action rinse. Thus, pyrethrin shampoos which are designed to be administered to an infested patient after wetting of the hair, may not kill lice consistently or effectively. Pyrethrins are thus widely regarded as being non-ovicidal, meaning they are not capable of killing the eggs.

In order to effectively kill lice eggs, an insecticide needs to make its way through the egg shell, a physical system designed to keep out a wide variety of chemical materials and retain water inside. The egg shell has a detachable cap that bears a number of air pores that act to exclude fluids but permit the passage of gases. It is through these pores that the developing louse embryo breathes, and by which suitable formulations can penetrate to kill the developing louse embryo. Thus, the problems of penetration are dependent on physical parameters that must be breached.

Generally, the more viscous a fluid is, and the greater the surface tension, the less chance of penetration into the pores. However, even alcoholic solutions, which have the advantage of a low wetting angle that should allow fluid to flow into the pores, are not free of problems because they can develop air bubbles that are larger than the pores themselves and thus subsequently hinder penetration to the chorionic membrane of the egg.

Aqueous lotions, cream rinses, and shampoos have too great a wetting angle for fluid to flow into the pores directly, and will only enter if appropriate excipients are included that will wet and flow more readily than the body of the formulation. Consequently, despite the bubbles of a shampoo's foam being small enough to enter the pores, they are inhibited by the aqueous medium.

The patent literature discloses several attempts to address the problem of human head lice. U.S. Pat. No. 4,927,813 to Bernstein discloses a composition and method for removing head lice nits from a human. The composition contains formic acid and is applied to the hair and scalp of a patient for a period of time to dissolve the nit cement and loosen nits attached to the hair. Although the composition may be effective at loosening attached nits, at higher preferred concentrations of efficacy, the composition can cause erythema and/or stinging or itching at the application site.

United States Patent Publication No. 2014/0296182 (182 Publication) to Levin et al. discloses methods and compositions for the treatment of pediculosis comprising specific amounts of isopropyl myristate, silicone oil, and water. The '182 Publication also discloses methods and compositions for dissolving nit glue comprising at least three components selected from the group consisting of benzyl alcohol, diacetone alcohol, acetic acid, and triethyl citrate. Despite the composition showing some efficacy in physically disrupting the waxy coating of the louse exoskeleton, and thus acting as a physical mechanism of action instead of a neurological mechanism of action, the presence of several living nymphs in some children after treatment indicates that the composition is not completely effective and requires repeated treatments at least after 7 days in cases of exceptionally serious infestations. Thus, the composition may not be effective in addressing all types of lice infestations, particularly those involving super lice.

International Publication No. WO 2010/134060 to Schwartz et al. discloses an all-natural, non-irritating pediculicidal composition comprising three components: a platform component, which acts essentially as a carrier; an aqueous phase component, which contains the primary active ingredients for killing the lice and for loosening the lice and nits from the hair (salt and vinegar, respectively); and an essential oil component, which contains additional active ingredients as well as ingredients for providing a pleasant scent. The composition uses an acetic acid concentration lower than that typically found (e.g., lower than 1%) in prior art acetic acid-containing pediculicidal compositions to purportedly achieve a synergistic effect among the ingredients of the composition through the joint action of acetic acid and salt to lessen the overall concentration of acetic acid needed to remove the lice effectively from the hair. However, because the active ingredients are primarily in the aqueous phase, the composition may suffer from too great a wetting angle for the pediculicidal to flow into the pores of the lice directly, and therefore, its efficacy may be inhibited by the aqueous medium itself.

Other prior art attempts at treating pediculosis have used chemical agents, including insecticides, to asphyxiate and/or paralyze the lice. These compounds include cholinesterase inhibitors such as neostigmine or prostigmine, and range from fairly toxic (e.g., pyrethrins) to very toxic (e.g., lindane, malathion). All methods involve the exposure of a person's scalp and hair to these agents in shampoos or creams which are subsequently washed off. Accordingly, long term safety and mitigation of side effects from use of pediculicidals relying on chemical agents are of concern to users of these products. Examples of prescription head lice treatments utilizing chemical agents include ULESFIA® (benzyl alcohol 5%) lotion, SKLICE® (ivermectin 0.5%) lotion, OVIDE® (malathion 0.5%) lotion, and Lindane 1% shampoo (Morton Grove Pharmaceuticals).

While current treatments may be effective at killing the adult lice and eggs (nits), the dead nits remain firmly attached to the hairs after treatment. This is unsettling to the patient, as well a significant cosmetic problem. The only current means of removing dead nits is with a fine-toothed comb or forceps, or incorporating formic acid, which can be irritating to the skin, into the pediculicide. These methods are all time consuming and less than certain.

### SUMMARY OF THE INVENTION

The inventor has identified a solution to the problems associated with current pediculicidal compositions. The solution resides in a composition effective at treating pediculosis by suffocation of lice. The composition is non-toxic, drug and pesticide free, paraben and/or formaldehyde free, and easy to administer to a patient suffering from head lice infestation. The composition comprises an oil blend, one or more terpene compounds, and an amount of a surfactant blend comprising an anionic emulsifier and an amphoteric emulsifier effective to suffocate lice. The amounts of the ingredients within the composition can vary (e.g., amounts can be as low as 0.000001% to as high as 98% w/w or any range therein). The combination of an oil blend, one or more terpene compounds, and an amount of a surfactant blend comprising an anionic emulsifier and an amphoteric emulsifier provides a composition which mitigates or reduces skin irritation and/or skin inflammation that would be present when applying pure essential oils to skin.

In some aspects, the composition includes a surfactant blend having an anionic emulsifier, an amphoteric emulsifier, and water. The combination can be used to create pediculicidal compositions that exhibit beneficial properties of decreasing surface tension at a louse body surface to promote impairment of breathing and/or suffocation of the lice, and/or prevent trapping of air pockets that the lice may use to breathe. In some instances, the surfactant blend serves as a primary emulsifier in combination with the primary oil base (e.g., coconut oil) to aid in creating a thick, tenacious emulsion capable of adhering to, smothering, and/or asphyxiating lice. In some instances, a small excess of surfactant is used above what is required for a typical emulsion to activate the surfactant properties including decreasing surface tension at a louse body surface, preventing air trapping, and/or reducing the time for asphyxiation. Unlike some emulsions which may be difficult to remove from the hair and scalp, this small excess of surfactant unexpectedly has the effect of creating a composition which can perform the benefits of suffocating lice, and then can be easily washed out of the hair. The composition including a small excess of surfactant exists as an emulsion and then dehydrates as it promotes impairment of breathing and/or suffocation of the lice. Then, when water is added, the composition surprisingly washes out of the hair and away from the scalp. The small excess of surfactant can include 1, 2, 3, 4, 5, 6, 7, 8, 9, 10, 11, 12, 13, 14, 15, 16, 17, 18, 19, 20 wt. % or more. In some instances, a small excess of surfactant can include 6 to 20 wt. % or more of surfactant. In some preferred instances, a small excess of surfactant can include 12 wt. % or more of surfactant.

In some aspects, the composition includes a primary oil base. In some aspects, the primary oil base is coconut oil. For example, the coconut oil can be virgin, cold pressed, refined, 76 degree, and/or fractionated coconut oil. In some aspects, the primary oil base may have a minimum melting point of −18° C. to 165° C., or any value within that range. In some particular embodiments, the primary oil base may have a minimum melting point of 60° C. to 100° C. Additionally, or alternatively, the primary oil base can include hemp seed oil, argan oil, shea butter, shea oil, sunflower seed oil, neem seed oil, jojoba oil, sweet almond oil, apricot kernel oil, avocado oil, amla oil, castor oil, evening primrose oil, flaxseed oil, grapeseed oil, kukui nut oil, macadamia oil, olive oil, sesame oil, wheatgerm oil and/or any other non-comedogenic oil.

In some aspects, the composition includes an oil blend having a primary oil base and a light oil base. In some aspects, the light oil base can include any light oil capable of coating hair without build up, weighing down hair, or clogging pores. Additionally, or alternatively, the light oil base can include any light oil capable of penetrating the hair shaft and/or moisturizing hair. For example, the light oil base can include any light oil (e.g., avocado oil), or combinations of light oils, having a molecular weight between around 150 g/mol to 300 g/mol. In some aspects, the light oil base is avocado oil. In some instances, the oil blend comprises 20 to 60 wt. % (e.g., greater than any one of, or between any two of: 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and/or 60) of a primary oil base and 1 to 25 wt. % (e.g., greater than any one of, or between any two of: 1, 2, 3, 4, 5, 6, 7, 8, 9, 10, 11, 12, 13, 14, 15, 16, 17, 18, 19, 20, 21, 22, 23, 24, and/or 25) of a light oil base. In some instances, the oil blend includes 30 to 50 wt. % of a primary oil base and 2 to 10 wt. % of a light oil base. In some instances, the oil blend includes 30 to 50 wt. % of coconut oil and 2 to 10 wt. % of avocado oil. The amounts of the primary oil base and/or light oil base can range from 0.0001% to 99.9% by weight or volume of the composition, or any integer or range in between as disclosed in other sections of this specification, which are incorporated into this paragraph by reference.

In some aspects, the surfactant blend includes 3 to 10 wt. % of an anionic emulsifier, 3 to 10 wt. % of an amphoteric emulsifier, and 30 to 45 wt. % of water. In some aspects, the surfactant blend includes 3 to 10 wt. % of sodium methyl cocoyl taurate, 3 to 10 wt. % of cocamidopropyl betaine, and 30 to 45 wt. % of water.

In some aspects, the composition includes 35 to 50 wt. % of the surfactant blend. In some instances, the composition is a cream. In some instances, the composition has a viscosity of about 1,000 cps to 100,000 cps at a temperature of between about 20° C. to 25° C. In some aspects, the composition will differ in viscosity based on the target hair type. In some such instances, the composition will have a lower viscosity for treatment for thick or wiry hair to ensure treatment is able to reach the scalp. In some instances, the composition has a viscosity of about 10,000 cps to about 50,000 cps at a temperature of between about 20° C. to 25° C. In some instances, the composition may exhibit a first viscosity state upon application and a second viscosity state after a duration of time post application. For example, a first viscosity state may be between about 8,000 cps to 95,000 cps upon application, and a second viscosity state (e.g., an anhydrous or substantially anhydrous viscosity state) may be between 800 cps to 8,000 cps after a duration of time (e.g., about 5, 10, 15, 20, 25, 30, 35, or 45 minutes after application) post application sufficient to promote water content in the composition to evaporate. In some aspects, the second viscosity state (e.g., an anhydrous or substantially anhydrous viscosity state) may be between 1,500 to 2,000 cps. Thus, in this way, the composition may exhibit beneficial properties in that the first viscosity state may contribute to saturating the area to be treated (e.g., hair, scalp, and roots of a person's head), and the second, lower viscosity state (e.g., the anhydrous or substantially anhydrous viscosity state) may provide the additional benefit of promoting the composition to further penetrate down to the target treatment area (e.g., the scalp of a person's head). Additionally, the second lower viscosity state (e.g., the anhydrous or substantially anhydrous viscosity state) may aid in the ease of rinsing out the composition after use, thereby providing an easier and more convenient way of removing the composition from the subject treatment area. Viscosity may be measured, for example, on a Brookfield Viscometer using a TC spindle at 2.5 rpm at 25° C., or other conventional methods known to those skilled in the art. The amounts of the surfactant blend can range from 0.0001% to 99.9% by weight or volume of the composition, or any integer or range in between as disclosed in other sections of this specification, which are incorporated into this paragraph by reference.

In some aspects, the composition is pesticide-free. In some aspects, the composition includes a paraben-free preservative system. In some instances, the paraben-free preservative system includes phenoxyethanol, caprylyl glycol, and sorbic acid. In some instances, the paraben-free preservative system includes 45 to 60 wt. % phenoxyethanol, 35 to 50 wt. % caprylyl glycol, and 2 to 10 wt. % sorbic acid. In some instances, the paraben-free preservative system is OPTIPHEN™ Plus, Ashland. In some instances, the composition includes 0.5 to 2 wt. % of the paraben-free preservative system.

In some aspects, the composition includes an essential oil or an essential oil blend. In some instances, the essential oil blend comprises peppermint oil, lavender oil, and/or tea tree oil. In some instances, the composition contains an effective amount of peppermint oil, lavender oil, and/or tea tree oil to increase activity and/or motility of lice. In some instances, the composition contains 0.01 to 1 wt. % peppermint oil, 0.001 to 1 wt. % lavender oil, and/or 0.001 to 1 wt. % tea tree oil. For example, the essential oil blend may comprise an effective amount of peppermint oil, lavender oil, and/or tea tree oil to act as a lice irritant, thereby increasing the activity and/or motility of the lice and causing the lice to become asphyxiated quicker as the composition penetrates the spiracles of the lice. In some instances, the lavender oil is lavender oil 40/42, a mixture of pure and synthetic lavender oil including linalool and linalyl acetate. In some aspects, the composition includes 1 to 100 wt. % peppermint oil, 1 to 100 wt. % lavender oil, and/or 1 to 100 wt. % tea tree oil.

In some instances, the composition further contains one or more of stearyl palmitate, glyceryl stearate, cetearyl alcohol, polyglyceryl oleate, and/or glyceryl oleate. In some instances, the composition contains one or more of 3 to 10 wt. % of stearyl palmitate, 1 to 8 wt. % of glyceryl stearate, 1 to 8 wt. % of cetearyl alcohol, 1 to 8 wt. % of polyglyceryl oleate, and/or 0.1 to 5 wt. % of glyceryl oleate.

In some instances, the composition further contains one or more terpene compounds. Non-limiting examples of terpene compounds include: linalool, limonene, citral, 1,8-cineole, anisole, β-pinene, menthone, α-pinene, pulegone, myrcene, geraniol, terpineol, hinokitiol, aucubin, catalpol, eugenol, carvone, methyl salicylate, terpinen-4-ol, carveol, nerolidol, thymol, α-terpineol, anethole, anisole, salicylaldehyde, benzaldehyde, cinnamaldehyde, benzyl cinnamate, or any combination thereof. In some aspects, the one or more terpene compounds contains linalool. In some aspects, the composition contains 0.00001 to 50 wt. % (e.g., greater than any one of, or between any two of: 0.00001, 0.0001, 0.001, 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and/or 50) of one or more terpene compounds. In some instances, the composition contains 0.00001 to 10 wt. % of one or more terpene compounds. The amount of the one or more terpene compounds can range from 0.0001% to 99.9% by weight or volume of the composition, or any integer or range in between as disclosed in other sections of this specification, which are incorporated into this paragraph by reference.

In some aspects, the compositions of the present invention can further include other ingredients identified in this specification or those known in the art. The composition can be a cream, wash, or emulsion (e.g., oil-in-water, water-in-oil, silicone-in-water, water-in-silicone, water-in-oil-in-water, oil-in-water-in-oil, oil-in-water-in-silicone, etc.). The composition can be formulated for topical application at least 1, 2, 3, 4, 5, 6, 7, or more times a day during use. In some aspects of the present invention, compositions can be storage stable or color stable, or both. It is also contemplated that the viscosity of the composition can be selected to achieve a desired result, e.g., depending on the type of composition desired, the viscosity of such composition can be from about 1 cps to well over 1 million cps or any range or integer derivable therein (e.g., 2 cps, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, cps, etc., as measured on a Brookfield Viscometer using a TC spindle at 2.5 rpm at 25° C.).

The compositions, in non-limiting aspects, can have a pH of about 6 to about 8 In some aspects, the pH can be 1, 2, 3, 4, 5, 6, 7, 8, 9, 10, 11, 12, 13, or 14. The compositions can also include preservatives. Non-limiting examples of preservatives include phenoxyethanol, caprylyl glycol, sorbic acid, or any mixture thereof. In some embodiments, the composition is paraben-free. In some embodiments, the composition is formaldehyde-free. In some embodiments, the composition is pesticide-free.

The compositions of the present invention can also include any one of, any combination of, or all of the following additional ingredients: a conditioning agent, a moisturizing agent, a pH adjuster, a structuring agent, inorganic salts, a preservative, a thickening agent, a silicone containing compound, an essential oil, a fragrance, a vitamin, a pharmaceutical ingredient, or an antioxidant, or any combination of such ingredients or mixtures of such ingredients. In certain aspects, the composition can include at least two, three, four, five, six, seven, eight, nine, ten, or more, or all of these additional ingredients identified in the previous sentence. Non-limiting examples of these additional ingredients are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference.

Methods of use for the compositions disclosed herein are also disclosed. In some aspects, a method is disclosed to treat pediculosis. In some instances, the method comprises applying to an affected area of a subject (e.g., head, hair, and/or pubic region) and/or the area in need thereof having pediculosis any one of the compositions (e.g., oil-in-water emulsion) disclosed herein, where application of any one of the compositions (e.g., oil-in-water emulsion) to the affected area suffocates lice present in the affected area to treat the pediculosis. In some instances, the lice to be suffocated include adult lice. In some instances, the lice to be suffocated include lice larvae and/or lice eggs (nits). In some aspects, any one of the compositions disclosed herein are topically applied and the composition is left on the application area, removed from the application area after a period of time, and/or removed directly after application.

In some implementations, the method includes topically applying to hair any one of the compositions disclosed herein. For example, the composition (e.g., an oil-in-water emulsion) may be applied to wet hair, damp hair, or dry hair that is infested with lice. In some instances, the hair has been towel dried prior to application of any one of the compositions disclosed herein. In some implementations, the compositions disclosed herein are topically applied to the entire scalp, hair, and/or roots. In some implementations, any one of the compositions disclosed herein is spread over an affected area of a subject and visually checked by spreading the hair in several places to ensure coverage from the hair down to the scalp and roots. In some implementations, the compositions disclosed herein are topically applied to the back of the head. In some implementations, hair is slicked backwards against the head after topical application of any one of the oil-in-water emulsions disclosed herein. In some implementations, longer hair is slicked backwards against the head and pulled upwards into a bun after topical application of any one of the compositions disclosed herein. In some implementations, any one of the compositions disclosed herein is worked into the hair and scalp, and then left in the hair and on the scalp after topical application to the affected area. In some implementations, the hair is rinsed thoroughly with water after any one of the compositions disclosed herein has been applied to the hair as a leave-on or rinse-off composition.

It is also contemplated that the compositions disclosed throughout this specification can be used as a leave-on or rinse-off composition. By way of example, a leave-on composition can be one that is topically applied to hair and remains on the hair for a period of time (e.g., at least 5, 6, 7, 8, 9, 10, 20, 30, or 45 minutes, or at least 1, 2, 3, 4, 5, 6, 7, 8, 9, 10, 11, 12, 13, 14, 15, 16, 17, 18, 19, 20, 21, 22, 23 or 24 hours, or overnight or throughout the day). In some embodiments, the leave-in composition is topically applied to hair and remains on the hair for at least 45 minutes. Alternatively, a rinse-off composition can be a product that is intended to be applied to the hair and then removed or rinsed from the hair (e.g., with water) within a period of time such as less than 5, 4, 3, 2, or 120 minutes. An example of a rinse off composition can be a shampoo, conditioner, or soap. An example of a leave-on composition can be a cream or leave-in conditioner.

In some implementations, the hair is then washed with shampoo and conditioner. In some implementations, the hair is then combed to remove any visible lice and/or nits. In some implementations, a detangler spray can be used with the combing step to aid in removal of any remaining lice and/or nits. In some implementations, the method of treating pediculosis is repeated every 3 days for a total of at least 3 treatments.

Kits that include any one of the compositions (e.g., oil-in-water emulsion) of the present invention are also contemplated. In certain embodiments, the composition (e.g., oil-in-water emulsion) is comprised in a container. The container can be a bottle, dispenser, or package. The container can dispense a pre-determined amount of the composition (e.g., oil-in-water emulsion). In certain aspects, the composition (e.g., oil-in-water emulsion) is dispensed in a dollop or liquid. The container can include indicia on its surface. The indicia can be a word, an abbreviation, a picture, or a symbol. In some embodiments, the kit includes any one of the compositions (e.g., oil-in-water emulsion) presently disclosed herein and a comb. In some embodiments, the kit further includes a head cap. In some embodiments, the kit further includes one or more gloves.

It is contemplated that any embodiment discussed in this specification can be implemented with respect to any method or composition of the invention, and vice versa. Furthermore, compositions of the invention can be used to achieve methods of the invention.

In some embodiments, compositions of the present invention can be pharmaceutically or cosmetically elegant or can have pleasant tactile properties. “Pharmaceutically elegant,” “cosmetically elegant,” and/or “pleasant tactile properties” describes a composition that has particular tactile properties which feel pleasant on the skin (e.g., compositions that are not too watery or greasy, compositions that have a silky texture, compositions that are non-tacky or sticky, etc.). Pharmaceutically or cosmetically elegant can also relate to the creaminess or lubricity properties of the composition or to the moisture retaining properties of the composition.

Also contemplated is a product comprising an oil-in-water emulsion of the present invention. In non-limiting aspects, the product can be a pediculicidal product. The pediculicidal product can be those described in other sections of this specification or those known to a person of skill in the art. Non-limiting examples of products include a cream, a shampoo, a conditioner, a wash, a body butter, a scrub, a cleanser, etc.

“Topical application” means to apply or spread a composition onto the hair, scalp, and/or roots of the hair. “Topical hair composition” includes compositions suitable for topical application on hair, scalp, and/or roots of the hair. Such compositions are typically dermatologically-acceptable in that they do not have undue toxicity, incompatibility, instability, allergic response, and the like, when applied to hair, scalp, and/or roots of the hair. Topical hair compositions of the present invention can have a selected viscosity to avoid significant dripping or pooling after application to hair, scalp, and/or roots of the hair.

“Lice” includes adult lice, lice larvae, and/or lice eggs (nits).

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations are refers to ranges within 10%, within 5%, within 1%, or within 0.5%.

The terms “inhibiting” or “reducing” or any variation of these terms includes any measurable decrease or complete inhibition to achieve a desired result. The terms “promote” or “increase” or any variation of these terms includes any measurable increase, such as a measurable increase of a molecule (e.g., evaporation of a water molecule) to achieve a desired result.

The term “effective,” as that term is used in the specification and/or claims, means adequate to accomplish a desired, expected, or intended result.

The use of the word “a” or “an” when used in conjunction with the terms “comprising,” “including,” “having,” or “containing,” or any variations of these terms, in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. With respect to the phrase “consisting essentially of,” a basic and novel property of the compositions and methods of the present invention is the ability to reduce the appearance of fine lines and wrinkles, moisturize skin, promote hydration, strengthen and repair skin, firm and condition skin, improve skin barrier function, reduce skin desquamation, reduce skin roughness, and increase skin lubricity.

Other objects, features, and advantages of the present invention will become apparent from the following detailed description. It should be understood, however, that the detailed description and the examples, while indicating specific embodiments of the invention, are given byway of illustration only. Additionally, it is contemplated that changes and modifications within the spirit and scope of the invention will become apparent to those skilled in the art from this detailed description.

### DESCRIPTION OF ILLUSTRATIVE EMBODIMENTS

As noted above, the present invention provides a solution to the problems associated with current pediculicidal products. The solution is premised on the use of a composition having a combination of ingredients capable of treating pediculosis by suffocation of lice, decreasing surface tension at a louse body surface, and/or preventing air trapping. In some instances, a combination of sodium methyl cocoyl taurate, cocamidopropyl betaine, and water can be used to create the pediculicidal compositions.

The following subsections describe non-limiting aspects of the present invention in further detail.

A particular composition of the present invention is designed to work as a topical hair composition. The composition relies on a unique combination of any one of, any combination of, or all of a surfactant blend containing an anionic surfactant and an amphoteric surfactant, an oil blend, and a preservative blend.

The surfactant blend includes an anionic surfactant (e.g., sodium methyl cocoyl taurate), an amphoteric surfactant (e.g., cocamidopropyl betaine), and water. In some aspects, the composition can be formulated as an emulsion including a surfactant blend containing 3 to 10 wt. % of an anionic surfactant, 3 to 10 wt. % of an amphoteric surfactant, and 30 to 45 wt. % water. In some aspects, the composition can be formulated as an emulsion including a surfactant blend containing 3 to 10 wt. % sodium methyl cocoyl taurate, 3 to 10 wt. % cocamidopropyl betaine, and 30 to 45 wt. % water. In this way, the surfactant blend serves as a primary emulsifier in combination with the primary oil base (e.g., coconut oil) and/or light oil base (e.g., avocado oil) to aid in creating a thick, tenacious emulsion capable of adhering to, smothering, and/or asphyxiating lice. In some instances, a small excess of surfactant may be used above what is required for a typical emulsion to activate the surfactant properties including decreasing surface tension at a louse body surface, preventing air trapping, and/or reducing the time for asphyxiation. In some instances, a small excess of surfactant unexpectedly has the effect of creating a composition which can suffocate lice, and then wash out of the hair with the addition of water. In some instances, a small excess of surfactant can include 6 to 20 wt. % or more of surfactant. In some preferred instances, a small excess of surfactant can include 12 wt. % or more of surfactant.

In some aspects, the composition can be formulated as an emulsion having a primary oil base and a light oil base, where the emulsion exhibits varying viscosity states at a temperature of between about 20° C. to 25° C. For example, the emulsion can have one, two, three, four, or more viscosity states depending on the environmental temperature and/or length of application of the emulsion. The different viscosity states may provide beneficial properties, such as, for example, promoting saturation of the treatment area (e.g., hair, scalp, and roots of a person's head), promoting penetration of the composition down to the target treatment area (e.g., the scalp of a person's head), and/or promoting penetration of the composition into the hair to moisturize the hair and promote release of lice from the hair. Additionally, the one, two, three, four, or more viscosity states may aid in the ease of rinsing out the composition after use, thereby providing an easier and more convenient way of removing the composition (e.g., an oil-in-water emulsion) from the subject treatment area.

Terpene compounds are aromatic compounds which are often components of many essential oils, plants, and flowers. In plants, terpenes are effective in defense against herbivores eating the plants, defense against disease, and attraction of pollinators. Terpene compounds can provide an anti-inflammatory effect on skin. Terpene compounds can be used to reduce pediculosis capitis by reducing the population of adult lice and reducing the development of lice eggs. As a non-limiting example, linalool, 1,8-cineole, anisole, limonene, β-pinene, menthone, α-pinene, pulegone, and myrcene provide a fumigant effect to adult head lice. As another non-limiting example, 1,8-cineole, anisole, anethole, limonene, α-pinene, β-pinene, and γ-terpinene reduce development of lice eggs. In some instances, the one or more terpene compounds are present in an essential oil.

In some aspects, the composition further contains one or more terpene compounds. In some aspects, the one or more terpene compounds includes at least one of: linalool, limonene, citral, 1,8-cineole, anisole, β-pinene, menthone, α-pinene, pulegone, myrcene, geraniol, terpineol, hinokitiol, aucubin, catalpol, eugenol, carvone, methyl salicylate, terpinen-4-ol, carveol, nerolidol, thymol, α-terpineol, anethole, anisole, salicylaldehyde, benzaldehyde, cinnamaldehyde, benzyl cinnamate, or any combination thereof. In some aspects, the one or more terpene compounds contains linalool. In some aspects, the composition contains 0.00001 to 50 wt. % of one or more terpene compounds. In some instances, the composition contains 0.00001 to 10 wt. % of one or more terpene compounds.

In some aspects, the preservative blend is pesticide free. In some aspects, the preservative blend is a paraben-free preservative blend containing phenoxyethanol, caprylyl glycol, and/or sorbic acid. In some aspects, the composition contains the preservative blend OPTIPHEN™ Plus (Ashland). In some instances, the paraben-free preservative system includes 45 to 60 wt. % phenoxyethanol, 35 to 50 wt. % caprylyl glycol, and 2 to 10 wt. % sorbic acid. In some instances, the paraben-free preservative system includes 50 to 55 wt. % phenoxyethanol, 40 to 45 wt. % caprylyl glycol, and 3 to 8 wt. % sorbic acid. In some instances, the composition includes 0.5 to 2 wt. % of the paraben-free preservative system. These combinations can be used to create topical hair compositions that treat pediculosis by decreasing surface tension at a louse body surface to promote impairment of breathing and/or suffocation of the lice, and/or prevent trapping of air pockets that the lice may use to breathe. Non-limiting examples of such compositions are provided in Tables 1 to 2 of Example 1 below.

Some compositions disclosed herein can be applied to and remain on the hair, scalp, and/or hair roots for a period of time (e.g., at least 1, 2, 3, 4, 5, 10, 20, 30, 45, or 60 minutes or more). After which, the composition, if needed, can be rinsed from the hair or combed from the hair. Some compositions disclosed herein can be applied to the hair, scalp, and/or hair roots and immediately rinsed. Some compositions disclosed herein can be applied to the hair and absorbed at least in part by the scalp and/or hair roots.

These and other non-limiting aspects of the present invention are described in the following sections.

### A. Active Ingredients

The present invention is premised on a determination that a combination of a surfactant blend containing an anionic surfactant and an amphoteric surfactant, and an oil blend including a primary oil base is effective at treating pediculosis, decreasing surface tension at a louse body surface, preventing air trapping, and/or reducing the time for lice asphyxiation.

In some aspects, the composition can be formulated as an emulsion containing sodium methyl cocoyl taurate as an anionic surfactant. Sodium methyl cocoyl taurate is the sodium salt of the coconut fatty acid amide of N-methyltaurine. In some instances, sodium methyl cocoyl taurate is commercially available, e.g., from Jarchem Innovative Ingredients, Newark NJ, which provides sodium methyl cocoyl taurate under the trade name Aminosyl™.

In some aspects, the composition can be formulated as an emulsion containing cocamidopropyl betaine as an amphoteric surfactant. Cocamidopropyl betaine is a mixture of closely related organic compounds derived from coconut oil and dimethylaminopropylamine. It has been determined that a combination of sodium methyl cocoyl taurate and cocamidopropyl betaine can be used to prevent air trapping, reduce the time for lice asphyxiation, and/or otherwise suffocate the lice. In some instances, this ingredient is commercially available, e.g., from Parchem Specialty Chemicals, New Rochelle, New York, which provides cocamidopropyl betain under the trade name Coco-betaine.

In some aspects, the oil blend includes a primary oil base and a light oil base. In some aspects, the light oil base is optional. In some aspects, the primary oil base is 76 degree coconut oil. Seventy-six degree (76° F.) coconut oil has a melting point of about 76 degrees fahrenheit, meaning the coconut oil is solid white at temperatures lower than 76 degrees fahrenheit. In some instances, the 76 degree coconut oil is commercially available, e.g., from Jedwards International Inc., Braintree MA, which provides 76 degree coconut oil under the trade name Refined Coconut Oil. In other aspects, the coconut oil can be virgin, cold pressed, refined, and/or fractionated coconut oil.

In some aspects, the light oil base is avocado oil. Avocado oil is commercially available, e.g., from Jedwards International Inc., Braintree MA, which provides avocado oil. In some aspects, the avocado oil can be United States Pharmaceutical grade avocado oil. In some aspects, the light oil base can include any light oil capable of coating hair without build up, weighing down hair, or clogging pores. In some instances, the avocado oil can be substituted with any one of a light oil base with a wide spectrum of molecular weights to allow for better consistency and/or emulsion stability.

In some aspects, the lavender oil is lavender oil 40/42. Lavender oil is commercially available, e.g., from Health & Beauty Natural Oils, Camarillo, CA, which provides lavender oil 40/42. In some aspects, the lavender oil base can include linalool and linalyl acetate. In some particular instances, the lavender oil 40/42 can include approximately 40% linalool and 42% linalyl acetate, respectively. In some instances, lavender 40/42 can include Lavandula angustifolia, Lavandula hybrida, Lavandula latifolia, lavandula stoechas, or any combination or singular lavender oil. In some instances, the lavender oil can be substituted with any one of an essential oil with a wide spectrum of molecular weights and smells to allow for better consistency and/or emulsion stability.

In some aspects, the composition further contains one or more terpene compounds. In some aspects, the one or more terpene compounds includes at least one of: linalool, limonene, citral, 1,8-cineole, anisole, β-pinene, menthone, α-pinene, pulegone, myrcene, geraniol, terpineol, hinokitiol, aucubin, catalpol, eugenol, carvone, methyl salicylate, terpinen-4-ol, carveol, nerolidol, thymol, α-terpineol, anethole, anisole, salicylaldehyde, benzaldehyde, cinnamaldehyde, benzyl cinnamate, or any combination thereof. In some aspects, the one or more terpene compounds contains linalool.

This combination of ingredients can be used in different product forms to treat pediculosis. By way of non-limiting examples, the combination of ingredients can be formulated in an emulsion (e.g., oil in water, water in oil), a gel, a gel emulsion, a scrub, a wash, a cream, or a body butter. In some instances, the product may relieve itching sensation on the scalp. In some instances, the product may produce a cooling sensation on the scalp.

### B. Amounts of Ingredients

It is contemplated that the compositions of the present invention can include any amount of the ingredients discussed in this specification. The compositions can also include any number of combinations of additional ingredients described throughout this specification (e.g., pigments, or additional cosmetic or pharmaceutical ingredients). The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

### C. Vehicles

The compositions of the present invention can include or be incorporated into all types of vehicles and carriers. The vehicle or carrier can be a pharmaceutically or dermatologically acceptable vehicle or carrier. Non-limiting examples of vehicles or carriers include water, glycerin, alcohol, oil, a silicon containing compound, a silicone compound, and wax. Variations and other appropriate vehicles will be apparent to the skilled artisan and are appropriate for use in the present invention. In certain aspects, the concentrations and combinations of the compounds, ingredients, and agents can be selected in such a way that the combinations are chemically compatible and do not form complexes which precipitate from the finished product. The compositions of the present invention can be structured or formulated into a variety of different forms. Non-limiting examples include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydro-alcoholic), gels, scrubs, body butters, peels, and ointments. Variations and other structures will be apparent to the skilled artisan and are appropriate for use in the present invention.

### D. Additional Ingredients

In addition to the combination of ingredients disclosed by the inventors, the compositions can also include additional ingredients such as cosmetic ingredients and pharmaceutical active ingredients. Non-limiting examples of these additional ingredients are described in the following subsections.

### 1. Cosmetic Ingredients

The CTFA International Cosmetic Ingredient Dictionary and Handbook (2004 and 2008) describes a wide variety of non-limiting cosmetic ingredients that can be used in the context of the present invention. Examples of these ingredient classes include: fragrance agents (artificial and natural; e.g., gluconic acid, phenoxyethanol, and triethanolamine), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), aroma agents (e.g., Stevia rebaudiana (sweetleaf) extract, and menthol), adsorbents, lubricants, solvents, moisturizers (including, e.g., emollients, humectants, film formers, occlusive agents, and agents that affect the natural moisturization mechanisms of the skin), water-repellants, essential oils, vitamins (e.g., A, B, C, D, E, and K), trace metals (e.g., zinc, calcium and selenium), anti-irritants (e.g., steroids and non-steroidal anti-inflammatories), botanical extracts (e.g., Aloe vera, chamomile, cucumber extract, Ginkgo biloba, ginseng, and rosemary), anti-microbial agents, chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), humectants (e.g., sorbitol, urea, methyl gluceth-20, saccharide isomerate, and mannitol), skin conditioning agents (e.g., aloe extracts, allantoin, bisabolol, ceramides, dimethicone, hyaluronic acid, biosaccharide gum-1, ethylhexylglycerin, pentylene glycol, hydrogenated polydecene, octyldodecyl oleate, and dipotassium glycyrrhizate). Non-limiting examples of some of these ingredients are provided in the following subsections.

a. Structuring Agents

In other non-limiting aspects, the compositions of the present invention can include additional structuring agents. Structuring agents, in certain aspects, assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

b. Emulsifiers

In certain aspects of the present invention, the compositions do not include an emulsifier. In other aspects, however, the compositions can include one or more emulsifiers. Emulsifiers can reduce the interfacial tension between phases and improve the formulation and stability of an emulsion. The emulsifiers can be nonionic, cationic, anionic, and zwitterionic emulsifiers (see U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, cetearyl glucoside, cetearyl alcohol, C12-13 pareth-3, PPG-2 methyl glucose ether distearate, PPG-5-ceteth-20, bis-PEG/PPG-20/20 dimethicone, ceteth-10, polysorbate 80, cetyl phosphate, potassium cetyl phosphate, diethanolamine cetyl phosphate, polysorbate 60, glyceryl stearate, PEG-100 stearate, arachidyl alcohol, arachidyl glucoside, and mixtures thereof.

c. Silicone Containing Compounds

In certain aspects of the present invention, the compositions do not include a silicone-containing compound. In non-limiting aspects, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

d. Essential Oils

Essential oils include oils derived from herbs, flowers, trees, and other plants. Such oils are typically present as tiny droplets between the plant's cells, and can be extracted by several method known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096. Pure essential oils, when applied directly to skin and/or with prolonged or repeated uses, can act as a skin irritant and/or cause skin inflammation.

Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present invention.

e. Thickening Agents

Thickening agents, including thickener or gelling agents, include substances which that can increase the viscosity of a composition. Thickeners includes those that can increase the viscosity of a composition without substantially modifying the efficacy of the active ingredient within the composition. Thickeners can also increase the stability of the compositions of the present invention. In certain aspects of the present invention, thickeners include hydrogenated polyisobutene, trihydroxystearin, ammonium acryloyldimethyltaurate/VP copolymer, or a mixture of them.

Non-limiting examples of additional thickening agents that can be used in the context of the present invention include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerytritol (e.g., Carbopol™ 900 series from B. F. Goodrich).

Non-limiting examples of crosslinked polyacrylate polymers include cationic and nonionic polymers. Examples are described in U.S. Pat. Nos. 5,100,660; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C10-C30 straight chain or branched chain alkyl group through an ether linkage. Typically these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

f. Preservatives

Non-limiting examples of preservatives that can be used in the context of the present invention include quaternary ammonium preservatives such as polyquaternium-1 and benzalkonium halides (e.g., benzalkonium chloride (“BAC”) and benzalkonium bromide), phenoxyethanol, benzyl alcohol, chlorobutanol, phenol, sorbic acid, thimerosal or combinations thereof.

### E. Kits

Kits are also contemplated as being used in certain aspects of the present invention. For instance, compositions of the present invention can be included in a kit. A kit can include a container, a comb, a head cap, and/or one or more gloves. Containers can include a bottle, a metal tube, a laminate tube, a plastic tube, a dispenser, a pressurized container, a barrier container, a package, a compartment, a compact container, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of the composition. In other embodiments, the container can be squeezed (e.g., metal, laminate, or plastic tube) to dispense a desired amount of the composition. The composition can be dispensed as a spray, an aerosol, a liquid, a fluid, or a semi-solid. The containers can have spray, pump, or squeeze mechanisms. A kit can also include instructions for employing the kit components as well the use of any other compositions included in the container. Instructions can include an explanation of how to apply, use, and maintain the compositions.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Exemplary Formulations

Formulations having the ingredients disclosed herein were prepared as pediculicidal compositions. The formulation in Table 1 is an example of a pediculicidal composition prepared as an oil-in-water emulsion. Both formulations work, but more of the mechanics of application—one just felt better than the other.

TABLE 1\hat{ }

| Ingredient | % Concentration (by weight) |
|---|---|
| Coconut oil - 76 degree melting point | 37.0 |
| Water | 36.4 |
| Cocamidopropyl betaine | 6.9 |
| Sodium methyl cocoyl taurate | 6.7 |
| Avocado oil | 6.0 |
| Stearyl palmitate | 6.0 |
| Glyceryl stearate | 3.8 |
| Polyglyceryl oleate | 3.0 |
| Cetearyl alcohol | 2.5 |
| Glyceryl oleate | 1.1 |
| Phenoxyethanol | 0.6 |
| Caprylyl glycol | 0.5 |
| Peppermint oil | 0.3 |
| Sorbic acid | 0.07 |
| Lavender oil 40/42 | 0.06 |
| Tea tree oil | 0.06 |
| Excipients* | q.s. |

\hat{ }The formulation can be prepared by mixing the ingredients in a beaker under heat at 70-75° C. until homogenous. Subsequently, the formulation can be cooled to standing room temperature (20-25° C.). Further, and if desired, additional ingredients can be added, for example, to modify the rheological properties of the composition or ingredients that provide additional benefits to hair and/or scalp suffering from head lice.
*Excipients can be added, for example, to modify the rheological properties of the composition. Alternatively, the amount of water can be varied so long as the amount of water in the composition is at least 20% w/w, and preferably between 30 to 45% w/w.

The formulation in Table 2 is another example of a pediculicidal composition prepared as an oil-in-water emulsion.

TABLE 2\hat{ }

| Ingredient | % Concentration (by weight) |
|---|---|
| Coconut oil - 76 degree melting point | 40.0 |
| Water | 21.0 |
| Avocado oil | 8.0 |
| Sodium methyl cocoyl taurate | 8.0 |
| Stearyl palmitate | 6.0 |
| Cocamidopropyl betaine | 4.0 |
| Glyceryl stearate | 3.5 |
| Sorbic acid | 3.0 |
| Cetearyl alcohol | 2.5 |
| Polyglyceryl oleate | 2.0 |
| Phenoxyethanol | 0.7 |
| Caprylyl glycol | 0.5 |
| Glyceryl oleate | 0.4 |
| Peppermint oil (optional) | 0.3 |
| Lavender oil 40/42 (optional) | 0.06 |
| Tea tree oil (optional) | 0.06 |
| Excipients* | q.s. |

\hat{ }The formulation can be prepared by mixing the ingredients in a beaker under heat at 70-75° C. until homogenous. Subsequently, the formulation can be cooled to standing room temperature (20-25° C.). Further, and if desired, additional ingredients can be added, for example, to modify the rheological properties of the composition or ingredients that provide additional benefits to hair and/or scalp suffering from head lice.
*Excipients can be added, for example, to modify the rheological properties of the composition. Alternatively, the amount of water can be varied so long as the amount of water in the composition is at least 15% w/w, and preferably between 20 to 45% w/w.

### Example 2

### Clinical Study Protocol

A controlled clinical study can be performed to evaluate the tolerability and efficacy of the head lice treatment. The study can take place over the course of nine days. On the first day, the scalp can be tested for head lice prior to test product usage. The test products participant can use will be either the formulation of Table 1 or Table 2. The treatment can include removal of the product by washing. Each participant will receive a total of three treatments, one every three days, and the participant or participant's representative will answer questions from a clinician or study personnel following each treatment.

Participants can be healthy volunteers, aged 2 years and up, who have head lice at the time of the clinical study. Participants can be tested for skin sensitivity by a medical professional on the first day prior to trial or can self-identify a level of skin sensitivity prior to trial, can be tested or self-identify skin sensitivity at the end of the clinical study. Volunteers who self-identify skin conditions such as eczema may be excluded from the clinical study to avoid skin irritation. Prior to taking part in the trial, participants or participants' representatives will provide the following information: contact information (e.g., participant name, participant address, representative name, relation to the participant, participant gender, contact phone number, email address, and preferred contacting method), participant allergies (e.g., to avocado, coconut, lavender, peppermint, tea tree), lice history and any previous product use, hair characteristics (e.g., thin, medium, or coarse, and straight, wavy, or curly), and hair length.

Additional information will be collected by a healthcare provider. Before treatment, a healthcare provider will provide an evaluation of the degree of infestation of lice (e.g., no presence, live lice, no live lice but nits less than ¼″ from scalp, nits present more than ¼″ from scalp), analyze whether skin inflammation is present on the scalp prior to treatment, obtain informed consent from the patient and/or patient's representative, provide instructions to patient and/or patient's representative, and provide the product to the patient to be tested. The healthcare provider may advise a volunteer with skin inflammation to not participate in the clinical study to avoid further skin irritation. Participants will be instructed to perform all three treatments regardless of whether it appears the lice are removed after the first treatment. Participants will wait two days between each treatment before continuing with the next treatment.

For each treatment, the treatment routine will include applying the test product to the hair and scalp, letting the test product dry for approximately 15 minutes to an hour, washing the product out of the hair with water, and then completing a survey and providing the results to the healthcare provider. The treatment routine can also include using a nit comb to comb out the lice and nits. The treatment can include using less than ½ a bottle of product, ½ a bottle of product, ¾ a bottle of product, one bottle of product, or more than one bottle of product. Participants can allow the product to dry for 15 minutes, 20 minutes, 25 minutes, 30 minutes, 35 minutes, 40 minutes, 45 minutes, 50 minutes, 55 minutes, one hour, more than one hour, or any time in between. Participants will record the amount of product used and the time they allowed it to dry before washing it out, and provide these numbers to the healthcare provider. Participants will also provide information to the healthcare provider related to how the scalp feels immediately following treatment (e.g., tingling, burning, soothing, cooling, or any combination), whether scalp itchiness was reduced, whether the Participant found the scent pleasant or unpleasant, and whether live lice were present after treatment.

After the final treatment, a healthcare provider will provide an evaluation of the degree of infestation of lice for each participant (e.g., no presence, live lice, no live lice but nits less than ¼″ from scalp, nits present more than ¼″ from scalp). Participants may also be asked to self-score for skin irritation.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for treating pediculosis, the method comprising applying to an affected area of a subject having pediculosis an oil-in-water emulsion comprising:
an oil blend; and
an effective amount of a surfactant blend comprising an anionic emulsifier and an amphoteric emulsifier,
wherein application of the emulsion to the affected area suffocates lice present in the affected area to treat the pediculosis.

2. The method of claim 1, wherein the oil-in-water emulsion further comprises one or more terpene compounds.

3. The method of claim 2, wherein at least one of the one or more terpene compounds is linalool.

4. The method of claim 1, wherein the surfactant blend comprises:
3 to 10 wt. % of the anionic emulsifier;
3 to 10 wt. % of the amphoteric emulsifier; and
30 to 45 wt. % of water.

5. The method of claim 1, wherein the surfactant blend comprises:
3 to 10 wt. % of sodium methyl cocoyl taurate;
3 to 10 wt. % of cocamidopropyl betaine; and
30 to 45 wt. % of water.

6. The method of claim 1, comprising 35 to 50 wt. % of the surfactant blend.

7-11. (canceled)

12. The method of claim 1, wherein the emulsion is pesticide-free.

13. The method of claim 1, wherein the emulsion comprises a paraben-free preservative system.

14. The method of claim 13, wherein the paraben-free preservative system comprises phenoxyethanol, caprylyl glycol, and sorbic acid.

15. The method of claim 13, wherein the emulsion comprises 0.5 to 2 wt. % of the paraben-free preservative system.

16. The method of claim 1, wherein the emulsion comprises an essential oil blend.

17. The method of claim 16, wherein the essential oil blend comprises peppermint oil, lavender oil, and/or tea tree oil.

18. The method of claim 17, wherein the emulsion contains an effective amount of peppermint oil, lavender oil, and/or tea tree oil to increase activity of lice.

19. An oil-in-water emulsion capable of treating pediculosis by suffocation of lice, the emulsion comprising:
an oil blend; and
an effective amount of a surfactant blend comprising an anionic emulsifier and an amphoteric emulsifier to suffocate lice.

20-22. (canceled)

23. The emulsion of any one of claims 19 to 22, wherein the surfactant blend comprises:
3 to 10 wt. % of sodium methyl cocoyl taurate;
3 to 10 wt. % of cocamidopropyl betaine; and
30 to 45 wt. % of water.

24. The emulsion of any one of claims 19 to 23, comprising 35 to 50 wt. % of the surfactant blend.

25-31. (canceled)

32. The emulsion of claim 19, wherein the emulsion comprises an essential oil blend.

33. The emulsion of claim 32, wherein the essential oil blend comprises peppermint oil, lavender oil, and/or tea tree oil.

34. The emulsion of claim 33, wherein the emulsion contains an effective amount of peppermint oil, lavender oil, and/or tea tree oil to increase activity of lice.

35. A kit comprising:
an emulsion of claim 1; and
a comb.

36. (canceled)

37. (canceled)
